(19) United States
(12) Patent Application Publication
(10) Pub. No.: US 2006/0079807 A1
(43) Pub. Date: Apr. 13, 2006

(54) Blood collecting syringe with retractable needle

(76) Inventor: Edward F. Allard, Springfield, VA (US)

Correspondence Address: Edward F. Allard, 7830 Greeley Blvd., Springfield, VA, 22152, US

(21) Appl. No.: 10/961,587

(22) Filed: Oct. 12, 2004

(51) Int. Cl. A61B 5/00; B65D 81/00

(52) U.S. Cl. 600/576

(57) ABSTRACT

A blood-taking syringe having a retractable needle, releasable upon demand, having a needle assembly, under tension from a compressed spring, with a needle holding head supporting a double ended needle axially aligned with the central axis of the syringe, which is held in equilibrium by a releasable wedge interposed between slots in the needle assembly and the inside of the syringe housing. A release pin extends through the side of the syringe at the point where the wedge is located and is disposed such that upon depressing the pin the wedge is dislodged from the slot in syringe housing, releasing the needle assembly to be forced into the syringe for safe storage.

### BACKGROUND OF THE INVENTION

1. Field of the Invention

This invention relates to blood-taking syringes, and more particularly to a syringe having a needle assembly which may, upon demand, be retracted into the housing of the syringe, in such a way that the needle becomes inaccessible, is rendered non-operational and the housing becomes a safe storage container for the spent needle and for any residual fluids.

2. Description of the Prior Art

The spread of diseases from the inadvertent exposure to contaminated needles has become a major concern throughout the health industry. Numerous attempts have been made to reduce the likelihood of the user being exposed to needle pricks and residual body fluids, by designing various schemes to cap the spent needle or to retract the needle into the cavity of the syringe, but none of the approaches have proven to be entirely satisfactory in providing an easily actuated mechanism for retracting and safely storing the needle in a fail-safe container that can then be safely handled without fear of being exposed to the spent needle and collected fluids. The instant technique and apparatus for collecting blood and safely disposing of the spent needle filly meets the existent needs of the health care worker and effectively overcomes all the safety concerns of the prior art devices.

### SUMMARY OF THE INVENTION

The general purpose of this invention is to provide an operationally simple, easy, safe and effective way to retract the needle assembly of a blood taking device, upon demand, into the syringe housing, to prevent the device from being reused, and then to seal the housing, rendering it a safe storage container for the needle assembly.

### BRIEF DESCRIPTION OF DRAWINGS

FIG. 1 shows a cut-away view of a blood-taking device as envisioned by this invention.

FIG. 2 shows the needle assembly of the blood-taking device in a retracted state.

### DESCRIPTION OF THE PREFERRED EMBODIMENTS

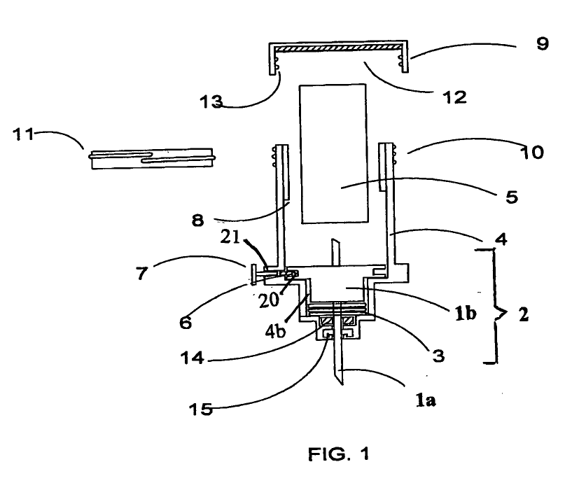

This invention will be best understood by referring to the drawings. Looking now at the blood taking syringe of FIG. 1, note the syringe housing (4) having first and second chambers, (4a) and 4(b), respectively, with the first chamber (4a) adapted to receive a blood taking vacuum vial (5) and with the second chamber (4b) designed to hold a spring loaded, double ended needle (1a) within a needle holder head (1b) of the hypodermic needle head assembly (2). The double ended needle (1a) is mounted within a needle head assembly (2) and extends axially therethrough, allowing the distal end of the needle to be inserted into a vein of a blood giver while the other end punctures one end of a vacuum vial (5) upon inserting the vial into chamber (4a) and inserting moderate pressure thereto. As is well known in the art, the vacuum within the vacuum vial (5) then draws blood from the vein of the patient and fills the vial accordingly. Any desired number of vials of blood may be drawn from the patient before initiating the withdrawal of the needle from the patient and releasing the needle head assemble (2) for storage within chamber (4a) of syringe housing (4). A compressed spring (3) tends to force the needle assembly (2) into the inside of the upper chamber (4a) of the syringe housing (4), however, spring (3) is prevented from driving the head assembly (2) into chamber (4a) by a holding wedge (6), which can be fabricated of hard plastic or vinyl or any other material strong enough to hold the needle assembly (2) in a state of equilibrium from the force of the compressed spring (3). After the desired number of vials of blood are drawn, the last vial may then be removed from the syringe and retraction of the needle head assembly (2) may be effected by depressing the release pin (7), which forces the end of pin (7) into the gap between the container holder (4a) and the needle assembly (2), making contact with the edge of the holding wedge (6), which is spring biased in groove (20) of the needle holder head (1b) to assure that the holding wedge (6) is held in an intermediate position within both the groove (20) and the slot (21) in syringe housing (4), until the release pin (7) is depressed to force the wedge (6) into groove (20) of the needle holder head (1b), thereby allowing the force of the compressed spring (3) to force the needle head assembly (2) into the chamber (4a) of the syringe for storage. A push-in motion applied to the push tab (7) by the thumb or finger releases the holding wedge (6) and can be performed easily with one hand. Even though not shown, another push tab and holding wedge may be used on the opposite side of the syringe housing from wedge (6), allowing the two push tabs to be squeezed simultaneously between the thumb and forefinger to release the needle assembly. The spring then would be free to expand and force the needle head assembly into the container holder until it is stopped by a protrusion on the inside of the housing (4), shown as a stop (8). This stop may be molded into the syringe housing (4) at manufacture or may be a simple sleeve inserted within the housing. The stop (8) may have an inwardly extending beveled edge at its most inwardly extending end. The beveled edge functions as a stop for stopping the upward movement of the needle head (2), for forcing the needle off center, and for providing a guide for vacuum vial (5). Once the needle assembly is retracted into the syringe housing chamber (4a), it cannot be reused. A safety cap (9) may be attached to the syringe housing, which is here shown as a screw-on type, but may be of any design that would securely seal the cap when applied to the container. The cap is twisted onto the guide (10). The safety cap may be similar to a push down, commercial safety cap used on safe liquid medicine bottles for protecting children. Removing the cap requires a push down force and a twist. The safety cap may seal (12) that seals in fluid due to the pressure created by the screw-on force. Safety cap (11) is one example, among many, that may be used to effect the sealing of the needle within the syringe. Broken rings (13) on the cap screw are twisted onto guide (10) on the housing. When the cap is screwed onto the housing, it seals that end of the housing, eliminating the possibility of the inadvertent removal of the cap and spilling contaminated fluid. Seal (14) is a self-sealing seal that immediately seals the hole left by the needle after retraction. This self-sealing material seals the lower end of the syringe housing, which prevents any inside contaminated fluid from leaking to the outside and prevents the retracted needle from protruding back through the hole left by retraction of the needle assembly (2). Cavity (15) captures any droplets of blood that might be wiped off the needle (1) by the seal (14) during retraction, however, an additional cap (not shown), similar to that of cap (9), may be used on the outermost end of the second chamber (4b) of the syringe housing (4), in the event that one desires to be assured that the spent needle and other body fluids are absolutely encapsulated within the housing upon retraction of the needle assembly. The two end caps (9) may be attached to the syringe upon manufacture, and shipped accordingly, in order to protect the needle from any potential environmental contamination.

Upon retraction of the needle assembly (2) into chamber (4a) of the syringe housing (4) and upon attaching safety caps (9) on the outermost ends of chambers (4a) and (4b) the needle head assembly and any contaminated fluids within the housing of the syringe (4) are encapsulated within the syringe housing (4), thereby rendering the syringe housing (4) a safe container for users and handlers who may come in contact with the used syringe.

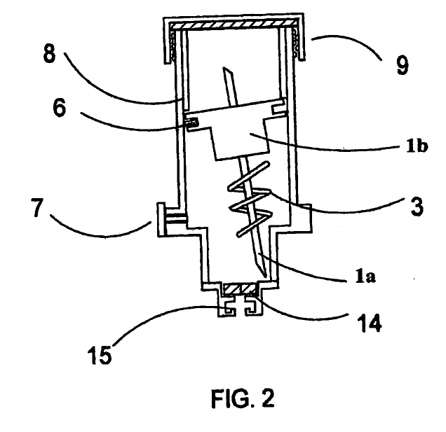

FIG. 2 shows the blood-taking device of FIG. 1 in its retracted state. Hypodermic needle (1), with the needle holder (1b) and the spring (3) has been retracted, been stopped by stop (8) and has been twisted off center. Push tab (7) has been pushed in, forcing holding pin (6) into needle head assembly (2), which has released spring (3). Seal (14) has automatically closed, sealing the bottom of the container. Safety cap (9) has been twisted onto the top of the container and sealed the top end accordingly. The container cannot be used again, thus the needle is inaccessible and the container is completely sealed, thereby rendering the used syringe (4) a safe container for the spent needle and fluids.

While this invention has been described in terms of a preferred embodiment, those skilled in the art will recognize that the invention can be practiced with modification within the spirit and scope of the appended claims.

Having thus described my invention, what I claim as new and desire to secure by Letters Patent is as follows:

1. A blood taking syringe comprising:
A syringe housing having a first chamber adapted to accept a removable blood-collecting vacuum vial and a second chamber adapted to hold a releasably mounted needle assembly under tension;
A needle assembly, including a cylindrically shaped needle retainer, having first and second ends, with a double-ended hypodermic needle extending through the center thereof with said needle and needle retainer being axially aligned with the central axis of the syringe housing;
A compressable spring, normally held in a compressed mode, seated in the second chamber of the syringe housing for applying pressure to the first end of the needle retainer along the central axis of the syringe housing;
A needle assembly holding means for applying a restraining force to the first end of the needle retainer for maintaining said needle assembly in a state of equilibrium;
Manually responsive pressure means for disengaging the needle assembly holding means to allow said needle assembly to be force into the first chamber of the syringe housing.

2. The blood-taking syringe of claim 1, wherein the needle retainer has a circumferential groove extending around the outer periphery of the second end thereof for accepting the needle assembly holding means and restraining the needle assembly to maintain a state of equilibrium.

3. The blood-taking syringe of claim 2, further including an outwardly extending protrusion on the syringe housing at the intersection of the first and second chambers of the syringe housing, with said protrusion having an inwardly open slot for accepting a portion of the needle assembly holding means, to allow the needle assembly holding means to simultaneously cooperate with both the groove in the second end of the needle retainer and the slot in the syringe protrusion, thereby holding the needle assembly in a state of equilibrium until released.

4. The blood-taking syringe of claim 3, wherein the needle assembly holding means is a dislodgable spring biased wedge interacting between the groove of the needle retainer and the slot in the syringe housing.

5. The blood-taking syringe of claim 4, wherein the groove around the periphery of the Second end of the needle retainer is of sufficient depth to accept the entire wedge upon disengagement and the slot within the syringe protrusion is of a depth sufficient to accept at least one half the width of the wedge, whereby the wedge is fully seated within the slot of the protrusion of the syringe housing while simultaneously extending into the groove of the needle retainer to hold the needle assembly in a state of equilibrium until manually released.

6. The blood-taking syringe of claim 5, wherein at least one hole extends through the wall of the housing, at the site of the slot of the syringe protrusion, for receiving at least one manually activated push-in tab, which upon activation, forces the portion of the dislodgable wedge lying within the slot in the syringe housing, fully onto the groove of the needle retainer, to release the needle assembly for retraction into the first chamber of the syringe housing.

7. The blood-taking syringe of claim 1, wherein the second chamber of the syringe housing is further adapted to seat a self-sealing seal through which the distal end of the double ended needle extends, such that after drawing blood and retraction of the needle and needle assembly into the first chamber of the syringe housing, the distal end of the syringe is sealed against the leakage of any fluid remaining within the body of the syringe.

8. The blood-taking syringe of claim 3, wherein the second chamber of the syringe housing is further adapted to seat a self-sealing seal through which the distal end of the double ended needle extends, such that after drawing blood and retraction of the needle and needle assembly into the first chamber of the syringe housing, the distal end of the syringe is sealed against the leakage of any fluids remaining within the body of the syringe.

9. The blood-taking syringe of claim 6, wherein the second chamber of the syringe housing is further adapted to seat a self-sealing seal through which the distal end of the double ended needle extends, such that after drawing blood and retraction of the needle and needle assembly into the first chamber of the syringe housing, the distal end of the syringe is sealed against the leakage of any fluids remaining within the body of the syringe.

10. The blood-taking syringe of claim 1, further having a fluid collecting cavity located below the self-sealing seal, in the distal end of the syringe, which collects any residual fluid wiped from the needle during the retraction of the needle through the self-sealing seal into the first chamber of the syringe housing, thereby preventing any residual fluid from leaking to the outside of the housing.

11. The blood-taking syringe of claim 3, further having a fluid collecting cavity located below the self-sealing seal, in the distal end of the syringe, which collects any residual fluid wiped from the needle during the retraction of the needle through the self-sealing seal into the first chamber of the syringe housing, thereby preventing any residual fluid from leaking to the outside of the housing.

12. The blood-taking syringe of claim 6, further having a fluid collecting cavity located below the self-sealing seal, in the distal end of the syringe, which collects any residual fluid wiped from the needle during the retraction of the needle through the self-sealing seal into the first chamber of the syringe housing, thereby preventing any residual fluid from leaking to the outside of the housing.

13. The blood-taking syringe of claim 9, further having a fluid collecting cavity located below the self-sealing seal, in the distal end of the syringe, which collects any residual fluid wiped from the needle during the retraction of the needle through the self-sealing seal into the first chamber of the syringe housing, thereby preventing any residual fluid from leaking to the outside of the housing.

14. The blood-taking syringe of claim 1, further having safety cap means for sealing both chambers of the syringe housing from the outside environment prior to use of the syringe and for resealing same upon use and retraction of the needle assembly.

15. The blood-taking syringe of claim 9, further having safety cap means for sealing both chambers of the syringe housing from the outside environment prior to use of the syringe and for resealing same upon use and retraction of the needle assembly.
